(19) United States
(12) Patent Application Publication
(10) Pub. No.: US 2012/0191176 A1
(43) Pub. Date: Jul. 26, 2012

(54) MEDICAL IMPLANT

(75) Inventors: Frank Nagl, Karlsruhe (DE); Werner Mailaender, Engelsbrand Grunbach (DE)

(73) Assignee: ACANDIS GMBH & CO. KG, Pfinztal (DE)

(21) Appl. No.: 13/496,047

(22) PCT Filed: Sep. 14, 2010
(86) PCT No.: PCT/EP2010/005625

§ 371 Date: Apr. 6, 2012

(30) Foreign Application Priority Data
Sep. 14, 2009 (DE) 10 2009 041 025.2

(51) Int. Cl. A61F 2/82

(52) U.S. Cl. 623/1.15

(57) ABSTRACT

A medical implant having a wall, which can be transferred from a compressed to an expanded state and includes a mesh structure formed of first struts, wherein the mesh structure has closed cells, each having a retaining element, which is adapted to anchor the expanded mesh structure in a vessel. The retaining element has at least two second struts which are connected to one another and form a tip which projects into the cell, and are connected to the first struts of the cell.

The invention relates to a medical implant with a wall, which comprises a mesh structure that can be transferred from a compressed state to an expanded state and that is formed by first struts, wherein the mesh structure has closed cells, each with a retaining element that is adapted to anchor the expanded mesh structure in a vessel. An implant of this kind is known from U.S. Pat. No. 5,397,355, for example.

In the field of interventional neuroradiology, two basic stent designs have proven useful for treating aneurysms and vascular stenoses, specifically closed-cell designs and open-cell designs. In recent times, closed-cell designs have increasingly been used, which have the advantage that a stent system that has already been partially deployed in the target area can be drawn back into the catheter. However, the commercially available stent systems, which are used in combination with coils for the treatment of aneurysms, can have weaknesses in terms of safety of use. After the stent system has been deployed, a coil catheter is guided through the cells of the stent into the aneurysm in order to bring the coil into the aneurysm. In doing this, the coil catheter can become caught in the tips of the cells, with the result that the stent can slip out of place if inadequately anchored in the blood vessel. Inadequate anchoring of the stent in the blood vessel can be caused, for example, by the outwardly directed radial force being too low or by the nature of the cell geometry. In addition, in known stent systems, weaknesses can often be observed in terms of the adaptation to the anatomical conditions existing in the neurocerebral vascular system.

The stent according to U.S. Pat. No. 5,397,355 has a mesh structure that is formed by struts and that can be transferred from a compressed state to an expanded state. The mesh structure is composed of closed cells (closed-cell design). In order to anchor the expanded mesh structure in a vessel, the cells each have a retaining element which, in the expanded state, is moved radially outward and protrudes beyond the wall of the stent. The retaining element is designed as a spike with a sharp tip which, in the implanted state, drills into the vessel wall. This can cause injuries.

Another possibility for anchoring a stent in the vessel is disclosed in U.S. Pat. No. 5,330,500 which, like the known stent described above, has hook-shaped tips in the cells, which tips drill into the vessel wall in the implanted state. U.S. Pat. No. 5,800,526 describes a stent with an anchoring system in which the mesh structure is designed such that parts thereof protrude beyond the wall of the stent in the implanted state and fix the stent against dislocation. However, the stent is not of a closed-cell design but of an open-cell design, which has the disadvantage that, after it has been deployed, it can be drawn back into the catheter only with difficulty. The same applies to the stent according to US 2006/0100695 A1, which has a mesh structure formed partially of closed cells and partially of open cells. The stent is a drug-eluting stent and for this purpose has an especially large surface area. To this end, various possibilities are disclosed, among which the possibility of connecting the struts of an open-cell stent portion via intermediate struts. These intermediate struts do not have the function of a retaining element and are instead intended solely to increase the effective surface area of the stent.

The object of the invention is to make available a medical implant that has a mesh structure with closed cells and that is safer to use.

According to the invention, this object is achieved by a medical implant with the features of claim 1.

The invention is based on the concept of making available a medical implant with a wall, which comprises a mesh structure that can be transferred from a compressed state to an expanded state and that is formed by first struts, wherein the mesh structure has closed cells, each with a retaining element. The retaining element is adapted to anchor the expanded mesh structure in a vessel. The retaining element has at least two second struts which, at one end, are connected to each other and form a tip, which projects into the cell. At the other end, the second struts of the retaining element are connected to the first struts of the cell.

The invention has several advantages. The configuration of the retaining element with at least two struts affords the possibility of an atraumatic design of the retaining element in contrast to the retaining element according to U.S. Pat. No. 5,397,355. In addition, the strut-shaped configuration of the retaining element has the effect that the number of struts on the circumference of the implant is increased, and therefore the radial force acting on the vessel wall is increased. This has the advantage that the safety of use is improved, particularly in respect of avoiding dislocation of the stent, without in so doing compromising the main advantage of closed-cell stent systems, namely the possibility of drawing the stent system back into the catheter after partial deployment in the target area.

In a preferred embodiment of the invention, the second struts of the retaining element form, with the first struts of a cell of the mesh structure, a further cell, which is arranged in the cell of the mesh structure. In addition to the increased radial force that can be achieved in this way, the arrangement of the cell within the cell has the effect that, when the implant curves as a result of a vessel shape, the tip of the retaining element is moved out beyond the wall into the vessel wall and securely anchors the implant.

The shape of the further cell can correspond to the shape of the cell of the mesh structure. This means that the smaller cell inscribed within the larger cell of the mesh structure and performing the retaining function repeats the shape of the larger cell. On the one hand, this has advantages from the point of view of production technology and, on the other hand, the deformation behavior of the further cell can be more easily predicted.

The first struts of the mesh structure can be connected to the second struts of the retaining element in each case between two connectors axially delimiting the struts. In particular, the first struts of the mesh structure can be connected centrally to the second struts of the retaining element. This permits a good transmission of force from the first struts of the cell to the second struts of the retaining element.

Alternatively, the second struts of the retaining element can be connected in each case to an axial end of the first struts of the mesh structure, wherein the retaining element replaces a connector axially delimiting the first struts. In this alternative, stress-optimized embodiment, the first struts, in particular the axial ends of the first struts, are therefore connected indirectly by the retaining element, which extends substantially into the cell of the mesh structure. The retaining element can also be lengthened into an area outside the cell of the mesh structure, such that the retaining element forms a further tip outside the cell, which further tip is coupled to an axially adjoining cell by a connector. In this embodiment, the retaining element is preferably symmetrical, with the corresponding axis of symmetry on the circumference of the mesh structure running through the axial ends of the first struts. In this way, the stress-optimized design of the structure is improved.

The longitudinal axis L′ of the further cell can be flush with a longitudinal axis L″ of the associated cell of the mesh structure, as a result of which the deflection of the tip of the retaining element leads to a good anchoring of the implant.

The tip of the retaining element can be arranged at least at the height of lateral connectors, which connect cells of the mesh structure that are arranged next to one another in the circumferential direction. This affords the possibility of the tip being deflected relatively far beyond the wall and thus being anchored correspondingly firmly to the vessel wall. The tip can point in the distal direction of the implant, which improves the possibility that the implant partially deployed in the vessel can be drawn back into the catheter. In a particularly preferred embodiment, at least one edge area, in particular two edge areas, and at least one intermediate area of the mesh structure are arranged in the longitudinal direction of the wall, wherein the retaining elements are formed in the edge area. This implant is particularly well suited for the treatment of aneurysms, since the anchoring function can be limited to the edge areas, and the intermediate area can assume the supporting and closing function, in order to close off the aneurysm, and coils located therein, with respect to the vessel lumen. The invention is not limited to this use. The division of the implant into edge areas and intermediate area permits a separation of functions that is also advantageous for other uses. The edge areas assume, or at least one edge area assumes, the retaining function on account of the retaining elements, and the intermediate area assumes another function that corresponds to the respective therapeutic purpose. It is also possible to provide the retaining elements in the intermediate area and to configure the edge areas for other functions.

In another preferred embodiment of the invention, the closed cells of the mesh structure form a plurality of ring segments arranged one after another in the axial direction of the wall, wherein the cells of a ring segment have at least one retaining element, in particular all the cells of the ring segment each have a retaining element. This affords the possibility of limiting the retaining function of the implant to a specific area, namely to one or more ring segments, and of adapting other areas of the implant to other functions.

The number of the distally arranged ring segments with retaining elements can be greater than the number of the proximally arranged ring segments with retaining elements. In this way, on the one hand, the implant is fixed securely in the vessel after being deployed, such that an exact positioning of the implant is possible. On the other hand, this improves the possibility of drawing the implant back into the catheter, since in the proximal area a smaller number of ring segments are provided with retaining elements.

It has proven particularly advantageous if the cell of the mesh structure comprises a diamond-shaped cell. The further cell, which is arranged in the cell of the mesh structure, can likewise comprise a diamond-shaped cell.

In a preferred embodiment of the implant, provision is made that the retaining element is adapted in such a way that, when the mesh structure curves along a longitudinal axis of the mesh structure, the tip of the retaining element automatically orients itself radially outward. In other words, provision is made that the retaining element protrudes radially outward in a curved or axially bent state of the mesh structure and can thus improve the anchoring in a vessel of the body.

A plurality of retaining elements are preferably provided which are distributed across the circumference of the mesh structure. When the mesh structure curves along a longitudinal axis of the mesh structure, the tips of the retaining elements can orient themselves radially outward, in particular only the tips of the retaining elements arranged on a side of the mesh structure directed away from the center of curvature. In the event of an axial curvature of the mesh structure, for example when the implant is arranged in a curve of a vessel, the retaining elements can orient themselves on one side, with the tips of the retaining elements being directed radially outward. The retaining elements preferably orient themselves on the side of the mesh structure directed away from the center of curvature. In particular, the retaining elements orient themselves radially outward on that side of the mesh structure which, in a curved or axially bent arrangement of the mesh structure, experiences a comparatively greater stretch. By contrast, the retaining elements arranged on an opposite side of the mesh structure, i.e. closer to the center of curvature, extend substantially in the wall plane and are not deflected radially outward. Generally, provision is made that the radially outwardly directed deflection of the retaining elements or of the tips of the retaining elements is caused by a curving of the mesh structure along a longitudinal axis or by an axial bending of the mesh structure.

In a preferred embodiment of the implant according to the invention, a plurality of retaining elements are provided, wherein the tips of all the retaining elements point in the same direction, in particular in the distal direction relative to a delivery system. Arranging the retaining elements to point in the same direction ensures that the implant can be drawn back into a delivery system without the tips of the retaining elements catching on the delivery system or on the vessel wall. Therefore, provision is particularly preferably made that the tips of all the retaining elements point in the distal direction, that is to say away from the person using the delivery system.

The invention is explained in more detail below on the basis of illustrative embodiments and with reference to the attached schematic drawings, in which:

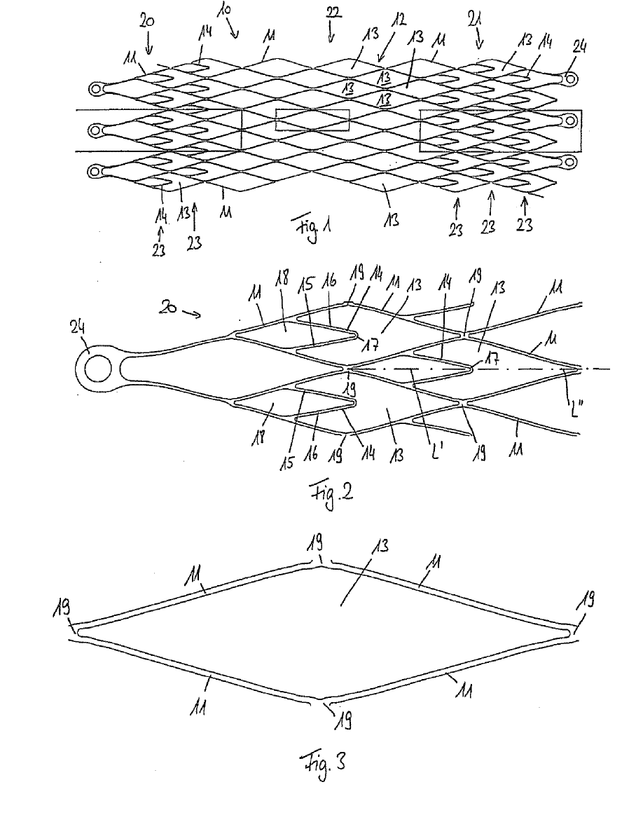

FIG. 1 shows a plan view of a stent, spread out in one plane, according to one illustrative embodiment of the invention;

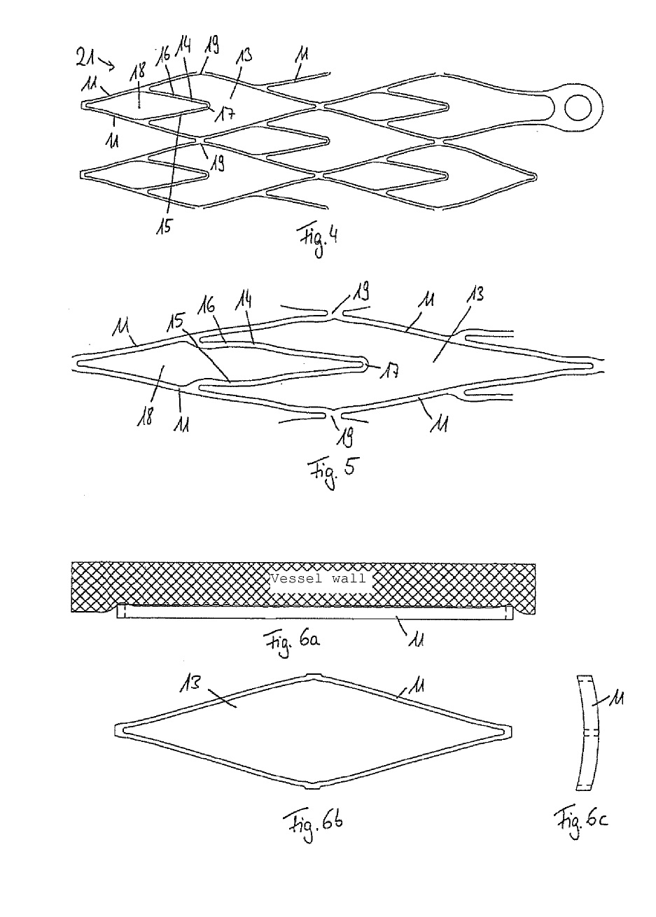

FIG. 2 shows a detail of the stent from FIG. 1 in the proximal edge area;

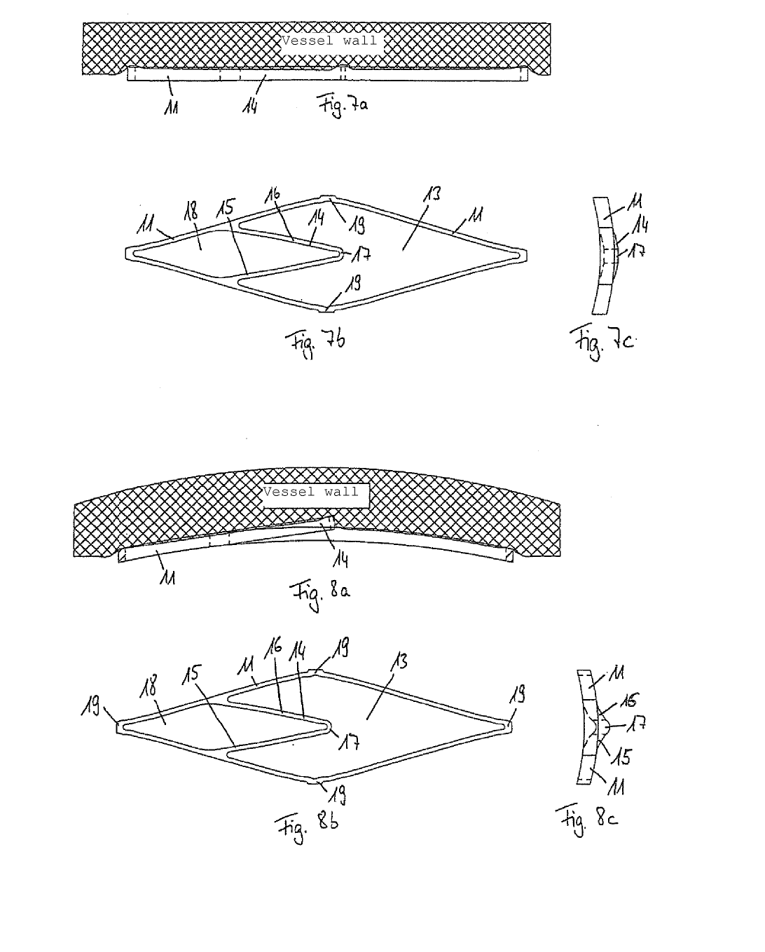

FIG. 3 shows a detail of the stent from FIG. 1 in the intermediate area 22, wherein a diamond-shaped closed cell is depicted;

FIG. 4 shows a detail of the stent from FIG. 1 in the distal edge area;

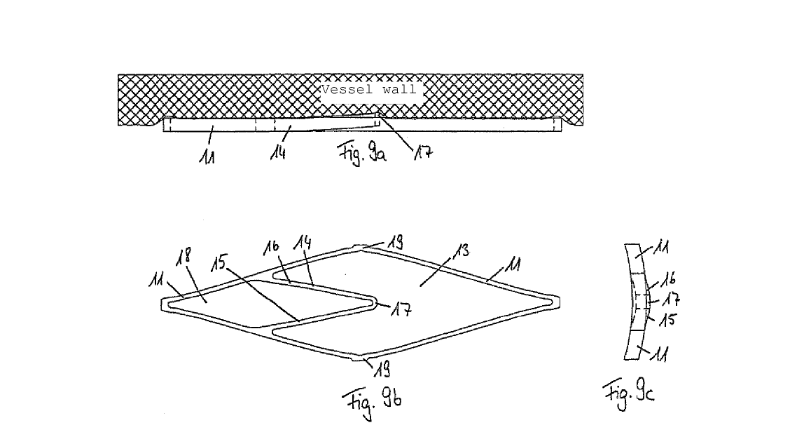

FIG. 5 shows a detail of the stent in the proximal or distal edge area during partial compression;

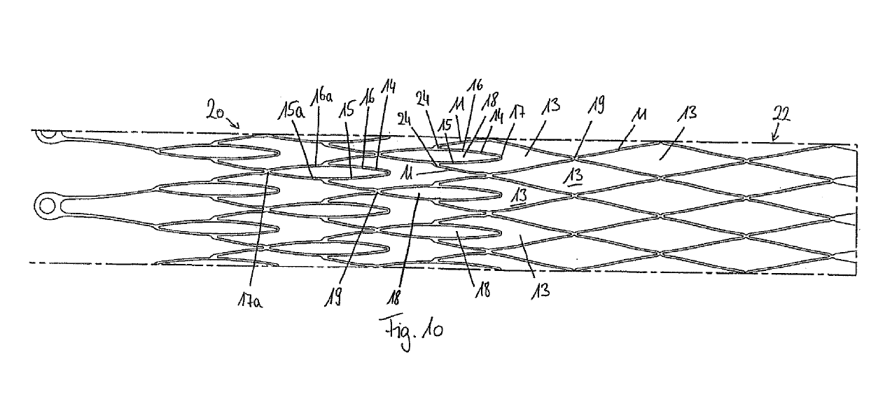

FIGS. 6a, 6b and 6c show views of the stent from FIG. 1 in the intermediate area in the implanted state;

FIGS. 7a, 7b and 7c show views of the stent from FIG. 1 in the distal or proximal edge area in the implanted state;

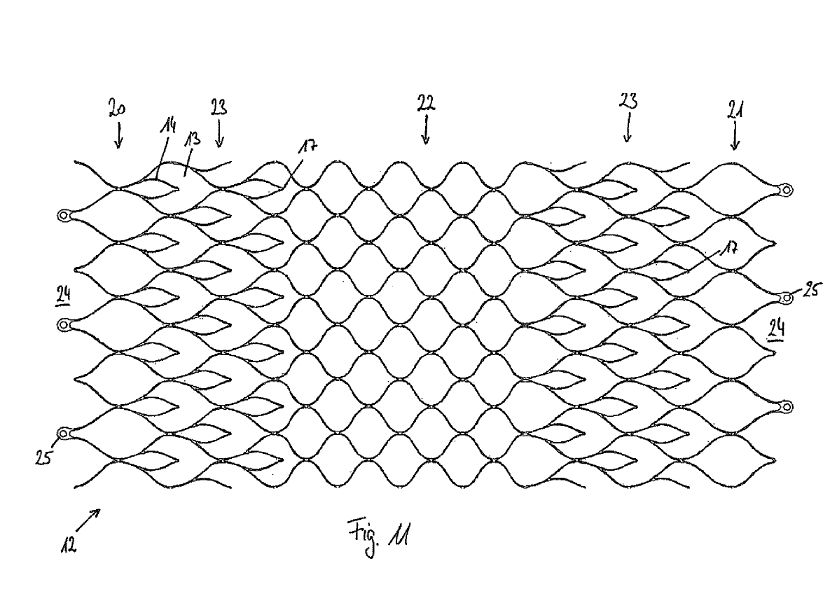

FIGS. 8a, 8b and 8c show views of the stent from FIG. 1 in the distal or proximal edge area in the implanted state, wherein the stent is curved;

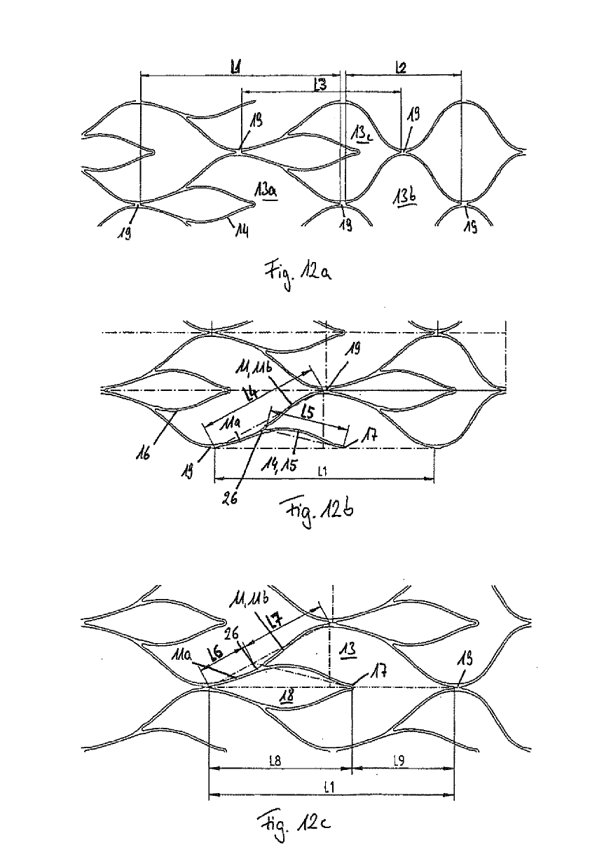

FIGS. 9a, 9b and 9c show views of a stent in the distal or proximal edge area in the implanted state according to a further illustrative embodiment of the invention;

FIG. 10 shows a plan view of a stent, spread out in one plane, according to a further illustrative embodiment of the invention;

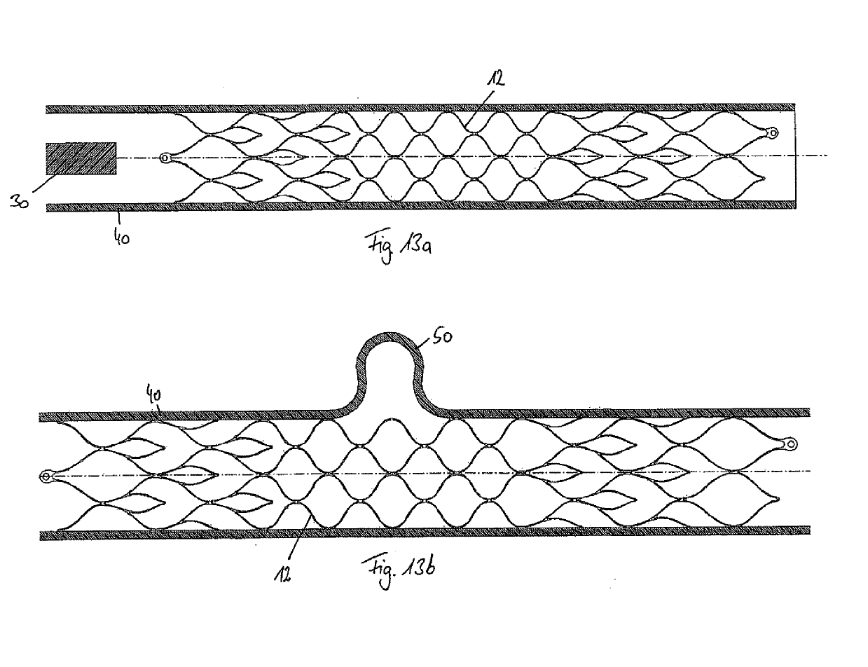

FIG. 11 shows a plan view of a stent, spread out in one plane, according to a further illustrative embodiment of the invention;

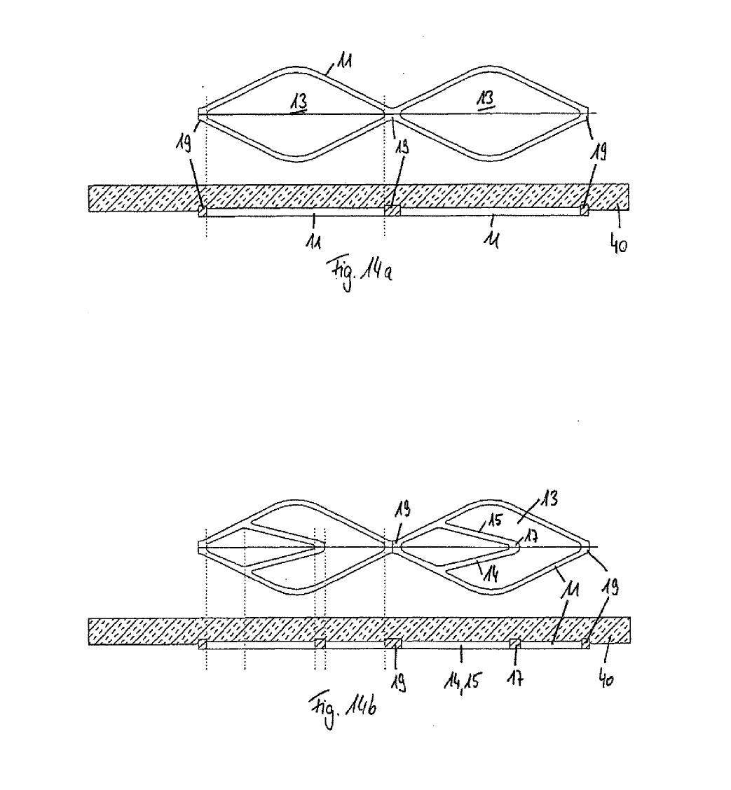

FIGS. 12a, 12b and 12c each show a detail of the stent from FIG. 11, wherein different length relationships are depicted;

FIGS. 13a and 13b each show a longitudinal cross section through a hollow organ of the body with an implanted stent from FIG. 11;

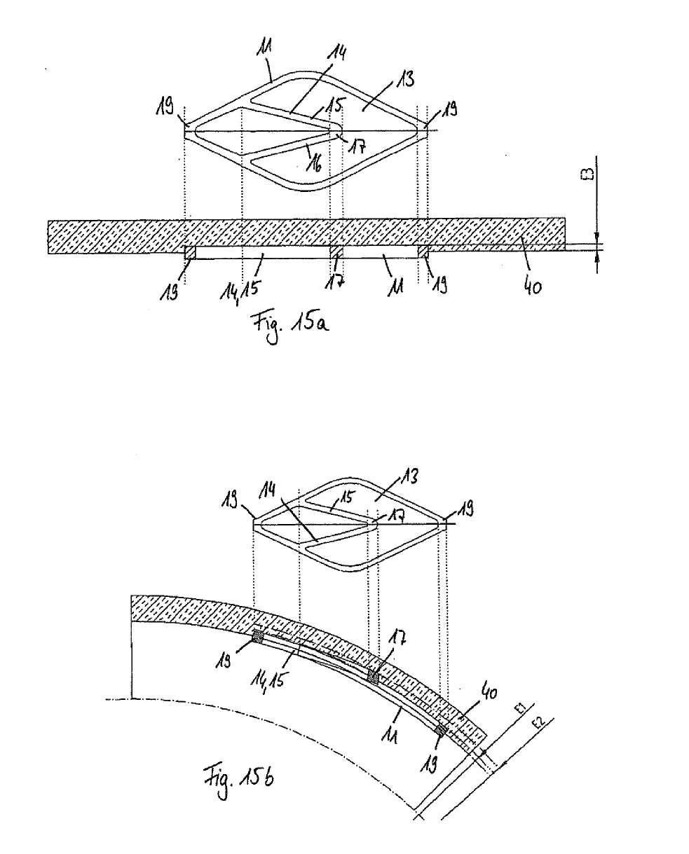

FIGS. 14a and 14b each show two views of two cells of a stent, wherein the anchoring of the struts or retaining elements of the stent in the vessel wall of a hollow organ of the body is depicted;

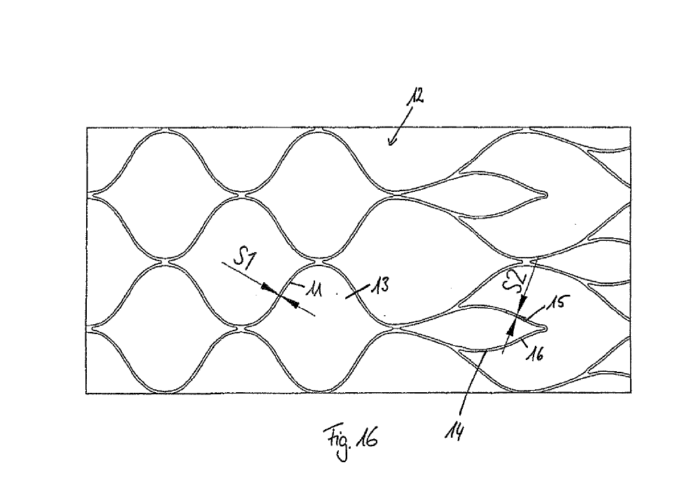

FIG. 15a shows two views of one cell of a stent with a retaining element, wherein the cell is arranged in a rectilinear hollow organ of the body;

FIG. 15b shows two views of one cell of a stent with a retaining means, wherein the cell is arranged in a curving hollow organ of the body; and

FIG. 16 shows a detail of the stent from FIG. 11, wherein the strut widths in different portions of the stent are depicted.

The stent shown in the figures is suitable, for example, for use in interventional neuroradiology, particularly for the treatment of aneurysms and vascular stenoses. The invention is not limited to stents and instead generally covers medical implants that are introduced into a hollow vessel of the body. The invention is also applicable, for example, to filters, flow separators or other medical implants. The implant, in particular the stent, can be self-expandable and can be produced, for example, from suitable materials such as nitinol or other shape-memory substances. The stent can also be designed to be expandable by balloon.

The implant or the stent comprises a wall 10 which, in the implanted state, comes into contact with the vessel wall and applies an outwardly acting radial force to the latter. The wall 10 comprises a mesh structure 12, which can be transferred from a compressed state to an expanded state. For this purpose, the mesh structure can be crimped in a manner known per se and, in the compressed state, can be loaded into a catheter. During implantation, the mesh structure 12 deploys and can be transferred into an expanded state.

The mesh structure is formed by first struts 11, which are produced, for example, by laser cutting, etching or other production techniques. The struts 11 of the mesh structure 12 form closed cells 13. In contrast to open cells, closed cells are fixedly connected to the adjoining neighboring cells by connectors 19.

As will be clearly seen from FIG. 2, a connector 19 connects two axially adjoining cells 13 and also two circumferentially adjoining cells. In this way, one connector 19 connects four cells 13 (except at the edge areas).

Thus, in contrast to an open cell, a closed cell forms a cell opening surrounded by struts 11, wherein the struts 11, at all the connection points to adjoining cells, are fixedly connected to these. Seen in the longitudinal direction of the stent, the connectors 19 form connection points that connect axially contiguous cells to one another. Seen in the circumferential direction, the connectors 19 likewise form connection points, which connect circumferentially contiguous cells fixedly to one another.

It is also possible that the mesh structure has closed and open cells.

Some of the closed cells 13 are designed with a retaining element 14, which is adapted to anchor the expanded mesh structure 12 in a vessel. It is also possible that a single cell 13 has a plurality of retaining elements 14. The retaining elements 14 are each formed from at least two second struts 15, 16 which, at one end, are connected to each other and have a tip 17. The tip 17 projects into the cell 13 of the mesh structure, as can be seen from FIG. 2. At the other end, the second struts 15, 16 are connected to the first struts 11 of the cell 13. This results in a strut-shaped retaining element, which has a V-shaped profile, wherein the tip 17 of the V-shaped profile points in the distal direction. The two struts 15, 16 diverge starting from the tip 17 and, at the end, are approximately as wide as the width of the struts 11 spanning the cell 13 in the area of the connection point. Another number of second struts 15, 16 is possible. The first struts 11 of the mesh structure 12, which are connected to the second struts 15, 16 of the retaining element 14, likewise have a V-shape, which is arranged in the opposite direction to the V-shape of the retaining element 14.

As is shown in FIG. 2, the second struts 15, 16 of the retaining element 14 form, together with the first struts of a cell 13 of the mesh structure 12, a further cell 18. The smaller cell 18 is arranged in the larger cell 13 of the mesh structure 12, wherein the smaller cell 18 is bordered partially by the second struts 15, of the retaining element and partially by strut portions 11 of the mesh structure. As can be clearly seen from FIG. 2, the shape of the further cell 18, i.e. of the smaller cell 18, corresponds to the shape of the cell 13 of the mesh structure 12 in which the further cell 18 is arranged. In the example according to FIG. 2, the cell is diamond-shaped in both cases. Other cell geometries are possible. The two second struts 15, 16 of the retaining element 14 engage approximately centrally on the first struts 11 of the cell 13.

It is also possible to connect the second struts 15, 16 of the retaining element 14 to the first struts 11 of the cell 13 at another, eccentric location, for example closer to the tip of the cell 13 of the mesh structure 12 or closer to the connectors 19. This means that the second struts 15, 16 of the retaining element 14 each engage at a position of the associated first struts 11 of the mesh structure 12 that is located between two connectors 19 axially delimiting the respective first strut 11. In this context, the term “axially” relates to the longitudinal extent of the individual strut. The retaining element 14 is therefore a separate element, which is provided additionally to the connectors 19. The tip 17 of the retaining element 14 is arranged at least at the height of the lateral connectors 19. In the illustrative embodiment according to FIG. 2, the tip 17 projects beyond an imaginary connecting line between the two connectors 19 of a cell. It is also possible to position the tip 17 behind the imaginary line between the two circumferentially arranged connectors 19. The tip 17 is rounded in order to make the anchoring of the implant as atraumatic as possible.

The stent design according to FIG. 2 is an axially symmetrical design with respect to the individual cells in which the further cells 18, formed by the struts 15, 16 of the retaining elements 14, and the cells of the mesh structure 12 lie on one line. Thus, the longitudinal axis L′ of the further cell 18 is flush with the longitudinal axis L″ of the respectively associated cell 13 of the mesh structure 12.

The tips of the individual retaining elements 14 each point in the distal direction, thus making reinsertion of the stent into the catheter easier.

As is shown in FIG. 1, some of the closed cells 13 of the mesh structure 12 have retaining elements 14. It is also possible for all the cells 13 to be provided with retaining elements 14. In the illustrative embodiment according to FIG. 1, two edge areas 20, 21 are provided in which the closed cells 13 have retaining elements 14. The intermediate area 22 arranged between the two edge areas 20, 21 is formed by cells 13 without retaining elements 14. Thus, the retaining elements 14 are arranged such that the middle area of the stent does not lose pore size, and the patency of the cells in the middle area is not impaired. The anchoring function is thus limited to the edge areas. Another arrangement of the functionally different areas of the stent is possible. Specifically, the closed cells 13 with the retaining elements 14 form ring segments 23, which are arranged one after another in the axial direction. The individual ring segments 23 are composed of circumferentially arranged closed cells, which are in each case connected to one another by lateral connectors 19. As can be seen from FIG. 1, the number of the proximally arranged ring segments 23 is greater than the number of the distally arranged ring segments 23, as a result of which precise positioning of the stent is achieved upon release, together with relatively simple retraction. In the illustrative embodiment according to FIG. 1, two ring segments 23 are provided at the proximal end of the stent, and three ring segments 23 are provided at the distal end of the stent. Another number of ring segments 23 with retaining elements 14 is possible both proximally and also distally. It is also possible for the same number of ring segments 23 with retaining elements 14 to be provided both proximally and distally.

The two edge areas 20, 21 are each connected to markers, in particular to X-ray markers 24.

The closed cells 13 of the mesh structure 12 have a diamond-shaped geometry, wherein the individual branches of the diamond geometry are formed by the struts 11 of the mesh structure. The shaping angle of the individual cell 13, i.e. the angle between the longitudinal axis of the cell and the connecting line between a lateral connector 19 and a connector 19 forming the tip of the cell, is preferably 50°. The shaping angle can be ≧25°, ≧30°, ≧35°, ≧40°. The upper limit of the shaping angle can be ≦60°, ≦55°, ≦50°, ≦45°. The above values of the shaping angle relate to the rest state.

FIG. 5 shows a partially deformed cell 13 with retaining element 14, which is deformed correspondingly to the cell 13.

The function of the invention is explained below with reference to FIGS. 6 to 8.

As is shown in FIGS. 6a, 6b and 6c, a purely closed-cell design, i.e. a closed cell 13 without retaining element 14, has the effect that the vessel wall is tensioned quite strongly, with the result that the struts 11 of the closed cell 13 press more weakly into the vessel wall. By contrast, as is shown in FIG. 9a, the retaining element 14 presses more strongly into the vessel wall, which retaining element 14, in the implanted state, partially protrudes radially outward past the wall of the stent or generally of the implant. Injuries to the vessel wall are avoided by the rounded atraumatic tip 17. The outwardly directed radial deflection of the retaining element 14 in the expanded state is also shown in FIG. 9c. The abovementioned effect can be strengthened by suitable dimensioning or geometric configuration of the retaining element 14, as is illustrated in FIGS. 9a, 9b and 9c. It will be seen from these that, even with a purely axial orientation of the stent, the retaining element 14 protrudes past the wall 10, such that the atraumatically shaped tip 17 is pressed farther into the vessel wall than the connectors 19.

It is also possible to design the retaining element 14 in such a way that the tip 17 does not protrude beyond the wall 10 in the expanded state but instead remains in the plane between the struts 11, as is shown in FIGS. 7a, 7b and 7c. The radial deflection of the retaining element 14 is set by the ratio of the strut width to the wall thickness of the struts 11. It is thus possible to influence the position of the tip 17 in the expanded state in such a way that said tip either remains in the wall 10 or protrudes outward past the wall 10.

In the case where the retaining element 14 is not deflected radially outward and the tip 17 remains in the wall plane, the retaining element 14 has a retaining function. The retaining function of the retaining element 14 arises in this case from the fact that the struts 11 and the retaining element 14 press into the intima of the vessel wall, as a result of which a corresponding resistance effect is produced.

The anchoring of the stent by means of the retaining elements 14 is particularly marked when the stent curves, as is shown in FIGS. 8a, 8b and 8c. Here, the retaining elements 14 are moved even more strongly out from the wall plane and press more deeply into the vessel wall. In this way, the risk of dislocation of the stent in the distal direction is reduced still further.

Another illustrative embodiment of the stent according to the invention is shown in the plan view according to FIG. 10. Here, a circumferential segment of the stent is shown in the deployed state, wherein the illustrated circumferential segment comprises a part of the intermediate area 22 and a circumferential portion of the edge area 20. The intermediate area 22 is formed by closed cells 13 which, in each case with four connectors 19, are coupled to adjoining cells 13 both in the axial direction and also in the circumferential direction. The closed cells 13 are substantially diamond-shaped.

In contrast to the closed cells 13 free of retaining elements in the intermediate area 22, closed cells 13 equipped with retaining elements 14 are arranged in the edge area 20. The retaining elements 14 are spanned between two first struts 11 of the mesh structure 12. In particular, the retaining elements 14 or the second struts 15, 16 of the retaining elements 14 are each coupled to axial ends 24 of the first struts 11. The axial end 24 of a first strut 11 thus forms the boundary of the strut 11 in the axial direction relative to the longitudinal extent of the individual strut 11 and merges directly into the connector 19. Analogously to the illustrative embodiments described above, the retaining element 14, in particular the second struts 15, 16 with the tip 17, forms a V-shaped profile that extends into the closed cell 13.

In the illustrative embodiment according to FIG. 10, the first struts 11 that are coupled to the retaining element 14 are shorter than the free first struts 11 that are connected directly to one another by connectors 19. Generally, therefore, the first struts 11 coupled to the retaining element 14 have a different axial length than the first struts 11 coupled directly or indirectly by connectors 19. It is also possible that the first struts 11 connected to the retaining element 14 have the same length as the free first struts 11.

As will also be seen from FIG. 10, the first struts 11 are articulated substantially on the second struts 15, 16, wherein the retaining element 14 extends between the first struts 11 in such a way that the retaining element 14 substantially replaces a connector 19 of the cell 13. In addition, the second struts 15, 16 are lengthened in the longitudinal direction of the stent and in each case form a strut continuation 15a, 16a. The strut continuations 15a, 16a converge and are brought together to form a common further tip 17a. Thus, the overall design of the retaining element 14 is symmetrical, wherein the axis of symmetry is fixed by the axial ends 24 of the first struts 11. The shape of the retaining element 14 can be described as being like a leaf spring or lens-shaped. The second struts 15, 16 thus each form an arc segment with the respective strut continuation 15a, 16a, wherein the arc segments are coupled to each other at the two longitudinal ends relative to the longitudinal axis of the stent and form the tip 17 or the further tip 17a.

Therefore, compared to the illustrative embodiments described above, in particular according to FIG. 2 and FIG. 5, the retaining element 14 is shifted in the direction of the closer stent end relative to the closed cell 13, such that the connection point between the first struts 11 and the second struts 15, 16 coincides with the position of the connector 19, which delimits the closed cell 13 in the longitudinal direction of the stent.

In an alternative interpretation of the illustrative embodiment according to FIG. 10, the closed cell 13 or main cell can be regarded as being formed by the first struts 11 and the strut continuations 15a, 16a. In this interpretation, the retaining element 14 is formed merely by the second struts 15, 16, which are guided together to a tip 17. The difference from the other illustrative embodiments described above is then that the first struts 11 are not arranged flush with the strut continuation 15a, 16a. Rather, the strut continuations 15a, 16a, which in this interpretation are regarded as part of the first struts 11, are oriented flush with the second struts 15, 16. Thus, at the connection point to the retaining element 14, the first struts 11 each form a kink or a curve, which forms the transition between the first struts 11 and the strut continuation 15a, 16a.

Independently of the interpretation used to describe the illustrative embodiment according to FIG. 10, the second struts 15, 16, together with the strut continuations 15a, 16a, particularly including the tip 17 and the further tip 17a, form a further, inner cell 18, which is arranged at least partially in the main cell or closed cell 13.

As will also be seen from FIG. 10, the shape of the first struts 11 in the intermediate area differs from the shape of the first struts 11 in the edge area. In the intermediate area, the first struts 11 have a substantially rectilinear design. This means that the connectors 19 in the intermediate area 22 are coupled by substantially straight or rectilinear first struts 11. Thus, between the connectors 19 in the intermediate area 22, the first struts 11 extend uncurved, at least in the circumferential plane of the stent. By contrast, in proximity to the connectors 19 delimiting the cell 13 in the longitudinal direction, the first struts 11 in the edge area have a curvature and/or form a thickened part in the area of the connector 19. In particular, the first struts 11 are oriented in a V-shape to each another, wherein the V-shaped tip ends in the area of the connector 19, and the angle between the first struts 11 varies, in particular widens as the distance from the connector 19 increases. It has been shown in general that a stress-optimized function can be achieved particularly with the structure of the stent according to the illustrative embodiment in FIG. 10.

The illustrative embodiments depicted in the figures are all based on a substantially diamond-shaped cell geometry, in which specially configured, in particular strut-shaped retaining elements 14 are arranged in the edge areas of the stent in such a way that the anchoring of the stent is improved and slipping of the stent during the intervention is avoided. The arrangement of the retaining elements 14 in the edge areas of the stent also has the result that in the middle area of the stent, i.e. in the area between the edge areas, the pore size is maintained, such that the patency of the cells in this area is not impaired. The arrangement of the tips 17 of the retaining elements in the distal direction leads to a barb effect being achieved, while at the same time it is possible for the retaining elements 14 of the stent, deployed to the extent of 80%, to be drawn back into a catheter. This is not possible in stents produced on the basis of an open-cell design.

FIG. 11 shows a stent in an illustrative embodiment according to the invention in the state when unwound. The stent according to FIG. 11 corresponds substantially to the stent from FIG. 1, but the first struts 11 have an S-shaped profile.

The stent according to FIG. 11 comprises two edge areas 20, 21, which are provided with X-ray markers 25. The edge areas 20, 21 each form the axial ends 24 of the stent. In particular, a row of circumferentially adjoining, closed cells 13 is provided at each of the axial ends 24 of the stent, wherein every second closed cell carries an X-ray marker 25. The edge areas 20, 21 are each adjoined by ring segments 23. In the illustrative embodiment according to FIG. 11, three rows of circumferentially adjoining, closed cells 13 are provided, which each form a ring segment 23. The ring segments 23 differ from the other segments of the stent in that the closed cells 13 in the ring segments each comprise a retaining element 14. Between the ring segments 23 there extends an intermediate area 22, which comprises one or more rows of circumferentially adjoining, closed cells 13. The closed cells 13 of the intermediate area 22 have a design free of retaining elements. In other words, the closed cells 13 of the intermediate area 22 have no retaining elements 14.

As will also be seen from FIG. 11, the tips 17 of the retaining elements 14 in the ring segments 23 each point in the same direction. It is particularly preferable if the tips 17 of all of the retaining elements 14 of the ring segments 23 or generally of the stent point in the same direction, particularly in the distal direction relative to a delivery system. In FIG. 11, the distal direction is to the right according to the drawing, whereas the proximal direction is to the left. In other words, the closed cells 13 of the edge area 21 in the right-hand part of the drawing form the distal end of the stent, whereas the closed cells 13 of the edge area 20 in the left-hand half of the drawing form the proximal end of the stent.

The closed cells 13 of the edge areas 20, 21, of the ring segments 23 and of the intermediate area 22 are preferably designed in such a way that the stent has a constant, in particular uniform, radial force along the entire length. Specifically, the shape and/or the size of the individual closed cells 13 in the respective segments or areas of the mesh structure 12 are designed in such a way that the standardized radial force, that is to say the radial force per stent length, or the radial pressure remains substantially constant along the entire length and circumference of the stent. For example, the closed cells 13 that comprise retaining elements 14 are larger than closed cells 13 that have no retaining elements 14, in order to compensate for the radial force increased by the retaining elements 14.

The closed cells 13 with retaining elements 14 in the edge areas 20, 21 of the mesh structure 12 can have a different radial force than the closed cells 13 of a central area, in particular of the ring segments 23 of the mesh structure 12 that have no retaining elements 14. The radial force can be influenced by the geometry of the cell 13. For example, the length of the cell 13, seen along the longitudinal axis of the mesh structure, or the angle of the first struts 11 of a cell 13 can be varied, in order to adjust the radial force in different portions of the mesh structure 12. The radial force in the edge area 20, 21 can be less than or greater than the radial force in a central area of the mesh structure 12. It is also possible that the cells in the edge areas 20, 21 are geometrically dimensioned in such a way that they have the same radial force as the cells 13 of a central area of the mesh structure 12. In particular, the radial force can be constant along the mesh structure 12. The adjustment of the radial forces along the mesh structure 12 is dependent on the use of the medical device and is chosen accordingly by the person skilled in the art. In doing so, account can be taken of the fact that a radial force in the edge areas 20, 21 of the mesh structure 12, where the cells 12 in the edge areas 20, 21 have retaining elements 14, reduces the risk of side effects, for example the danger of stenosis, in the edge area 20, 21 of the implanted medical device.

Generally, the deformation force of the respectively associated cell 13 is increased by the retaining elements 14. In the event of a radial compression of the stent or of the mesh structure 12 in relation to the rest state, not only the first struts 11 of the cell deform, but also the second struts 15, 16 of the retaining element. Compared to a cell that is designed free of retaining elements, i.e. has no retaining element 14, the radial force is increased by the presence of a retaining element 14 even if the cell 13 has the same geometric dimensions.

It is possible that the cells 13 in an edge area 20, 21 of the mesh structure 12 comprise anchoring cells 13a, i.e. cells 13 with retaining elements 14, which have the same profile and the same dimension ratios (strut width, strut length, cell angle, cell width, etc.) as the cells 13 free of retaining elements, i.e. the free cells 13b, in a central area (without retaining element). In this case, the radial force in the edge area 20, 21 is greater than in the middle area because of the additional retaining elements 14.

It may be advantageous if the radial force in the edge area 20, 21 of the mesh structure 12 is reduced or matched to the radial force in the central area of the mesh structure 12. In this way, a gentle transition, with respect to the mechanical properties of the cells 13, is achieved between the central area (free cells 13b) and the edge areas (anchoring cells 13a). In particular, the mechanical properties, particularly the radial force, of the edge areas 20, 21 and of the middle area can be identical. An abrupt transition characterized by very different forces can lead to considerable local strain on the vessel in the transition area, since the pulsatility wave or deformation wave of the vessel, which is caused by the pulsating blood flow, can change considerably on both sides of the transition area.

In a preferred embodiment, the cells 13 in the edge area 20, 21 have a different geometrical shape than the cells 13 in the central area. This has the effect that a cell 13 arranged in the edge area 20, 21 has per se, that is to say ignoring the retaining element, a lower radial force than a cell 13 in the central area of the mesh structure 12. This is achieved by, among other things, a smaller tilt angle, i.e. the angle between two first struts in the area of a connector, and/or by a smaller strut width, and/or by longer struts, and/or by a smaller number of cells 13 in a circumferential row, and/or by different strut geometries, in particular different radii of curvature of the first struts. The angle of the first struts 11 of the cells 13 in a central area of the mesh structure 12 can be smaller, preferably by at most 15°, in particular at most 10°, in particular at most 5°, than the angle of the first struts 11 of the cells 13 in an edge area 20, 21 of the mesh structure 12. This counteracts the effect of the increase in radial force by the retaining elements 14.

Overall, the following different possibilities are provided:

It is possible that the radial force in an edge area 20, 21 of the mesh structure 12 (cells 13 with retaining elements) is greater than in a central area of the mesh structure 12. A changing geometry of the cells 13 along the mesh structure 12 or the stent, particularly in the edge area, causes the radial force to increase in steps and/or gradually. An abrupt transition in the intermediate area 22 is thereby avoided.

It is also possible that the radial force in an edge area 20, 21 (cells 13 with retaining elements) is matched by the change of geometry to the mechanical properties, in particular the radial force, in a central area or is set to the same value. For example, only one or more first circumferential rows or one or more first circumferential segments of the mesh structure 12, which comprise anchoring cells 13a and are connected to free cells 13b of a central area, can have the same radial force as the circumferential rows or circumferential segments of a central area of the mesh structure 12. In further edge segments or edge areas, which are separate or spaced apart from the anchoring cells 13a of the central area, the radial force can increase gradually.

There is also the possibility that circumferential rows or circumferential segments which are arranged in an edge area 20, 21 and have cells 13 with retaining elements (anchoring cells 13a), or at least some of these circumferential segments with anchoring cells 13a, have a lower radial force than circumferential segments in a central area of the mesh structure 12, which have no retaining elements 14 or are formed by free cells 13b. In this way, the radial force in the edge area 20, 21 of the mesh structure 12 or of the stent is reduced. In particular, the radial force in the edge area 20, 21 of the mesh structure 12 can be lowered in steps and/or gradually in the direction of the axial end of the mesh structure 12, in order to protect the vessel walls in the implanted state, at least in those sections in which the edge area 20, 21 of the mesh structure 12 is arranged.

A combination of cells 13 and circumferential segments or circumferential rows of the edge areas 20, 21 with a lower radial force, the same radial force or a higher radial force compared to the cells 13 of a central area of the mesh structure 12 is possible.

The reduction of the radial force in the circumferential segments which comprise cells 13 with retaining elements is possible in view of the adherence of the stent or of the mesh structure 12 to a vessel wall, since the retaining elements 14 permit a “geometric” locking or adherence. This means that the locking action is provided at least in the main by the geometric shape of the retaining elements 14. The shape of the retaining elements 14 permits good adherence, even with a comparatively low radial force of the associated cells.

The radial force can be measured using conventional radial force measurement systems comprising a plurality of blades that close inward like a shutter. It is also possible to test separate cells 13. The separate cells 13 can be stretched or pressed on a tensioning machine. From the value of the deformation force, it is possible to deduce the value of the radial force of the stent.

Overall, the mesh structure 12 has rows S_{HE} of closed cells 13 with retaining elements 14 and rows S of closed cells 13 without retaining elements 14.

Preferably, two, three, four, five, six, seven, eight or nine rows S_{HE} of closed cells 13 with retaining elements 14 are provided in each of the axial end portions of the stent or of the mesh structure 12. Overall, the ratio between rows S_{HE} of closed cells 13 with retaining elements 14 to rows S of closed cells 13 without retaining elements 14 (S_{HE}/S) can cover a range of 2/4 to 2/10, in particular 3/3 to 3/12, in particular 4/4 to 4/10, in particular 5/5 to 5/15, in particular 6/4 to 6/16, in particular 7/5 to 7/24, in particular 8/4 to 8/20, in particular 9/5 to 9/27, in particular 10/4 to 10/30, in particular 12/4 to 12/32.

The closed cells 13 that have a retaining element 14 are referred to below as anchoring cells 13a. Closed cells 13 that have no retaining element 14 are called free cells 13b. Moreover, there are also transition cells 13c, which are each arranged between a row of anchoring cells 13a and a row of free cells 13b and provide the transition between the different dimensions of the anchoring cells 13a and the free cells 13b. The transition cells 13c can have retaining elements 14.

Different lengths and distances within the mesh structure 12 are shown in FIG. 12a. Here, the reference signs L1 to L8 designate the following lengths or distances:

As is shown in FIG. 12a, an anchoring-cell length L1 corresponds to the distance between two connectors 19 of an anchoring cell 13a. The connectors 19 in each case connect at least two first struts 11 of the anchoring cell 13a. The free cells 13b have a free-cell length L2 corresponding to the distance between two connectors 19 of the free cell 13b. The free-cell length L2 is preferably smaller than the anchoring-cell length L1. The transition cell 13c further comprises a transition-cell length L3, which likewise corresponds to the distance between two connectors 19 of the transition cell 13c. The transition-cell length L3 is preferably greater than the free-cell length L2 and smaller than the anchoring-cell length L1. The anchoring-cell length L1, the free-cell length L2 and the transition-cell length L3 relate in each case to the distance between two connectors 19 that are arranged adjacent, in particular in alignment, in the longitudinal direction of the mesh structure 12.

According to FIG. 12b, a connector spacing L4 can also be determined, which corresponds to the distance between two connectors 19 that are coupled by a common first strut 11. The connector spacing L4 thus extends substantially obliquely with respect to the longitudinal axis or the longitudinal direction of the mesh structure 12. From FIG. 12b, a tip spacing L5 can also be seen, which corresponds to the distance of the tip 17 of the retaining element 14 from a start point 26. The tip 17 and the start point 26 each form an end of the second strut 15, 16 of the retaining element 14. The start point 26 thus corresponds to the location where the second strut 15, 16 of the retaining element 14 originates from the first strut 11 of the closed cell 13.

Moreover, according to FIG. 12c, a start spacing L6 is provided, which corresponds substantially to the distance between a start point 26 and a connector 19, wherein the connector 19 is assigned to the further cell 18, to which the start point 26 also belongs. The further cell 18 is formed by two strut start portions 11a of two first struts 11, which are coupled in the connector 19, and the second struts 15, 16 of the retaining element 14. In other words, the retaining element 14, together with in each case one strut start portion 11a of two first struts 11, forms the further cell 18, which protrudes into the closed cell 13. The start point 26 is at a residual strut spacing L7 from a further connector 19 which is connected to the start point 26 by a residual strut portion 11b. The residual strut spacing L7 thus corresponds substantially to the distance between the start point 26 and a connector 19, wherein the distance is measured along the residual strut portion 11b. Overall, the first struts 11 from which the second struts 15, 16 of the retaining element 14 originate are divided into two portions, namely the strut start portion 11a and the residual strut portion 11b. The strut start portion 11a is part of the further cell 18 formed by the retaining element 14. By contrast, the residual strut portion 11b is assigned to the closed cell 13.

The distance between the tip 17 of the retaining element 14 and a connector 19, which is assigned to the further cell 18 formed by the retaining element 14, corresponds to the retaining element length L8, as is shown in FIG. 12c. In other words, the length of the further cells 18 in the axial direction or in the longitudinal direction of the mesh structure 12 is designated as the retaining element length L8. The tip 17 of the retaining element 14 is additionally aligned with a further connector 19 of the closed cell 13. The distance between the tip 17 and a further connector 19, which is aligned with the tip 17 in the longitudinal direction of the mesh structure 12 and is not assigned to the retaining element 14 to which the tip 17 is assigned, is designated as the opening length L9. The opening length L9 corresponds to the difference between the anchoring-cell length L1 and the retaining element length L8.

In this connection, it will be noted that the aforementioned lengths and spacings L4-L9, with the exception of anchoring-cell length L1, free-cell length L2 and transition-cell length L3, can relate in principle to the dimensions of all the closed cells 13. The lengths and spacings indicated are therefore not limited to a particular shape of the cell, i.e. not limited to an anchoring cell 13, a free cell 13b or a transition cell 13c. The spacings L4-L7 are in principle based on a rectilinear connection, shown in the figures as a dot-and-dash line. It will also be noted that all the lengths and spacings indicated relate to the fully expanded state of the mesh structure 12, i.e. the production state.

The following length and spacing ratios are preferred:

The ratio between the anchoring-cell length L1 and the free-cell length L2 (L1/L2) is preferably at least 0.7, in particular at least 0.8, in particular at least 0.9, in particular at least 1. Preferably, the ratio between anchoring-cell length L1 and free-cell length L2 (L1/L2) is at most 2.5, in particular at most 2.4, in particular at most 2.3, in particular at most 2.2, in particular at most 2.1, in particular at most 2.

The ratio between the transition-cell length L3 and the free-cell length L2 (L3/L2) is preferably at least 0.6, in particular at least 0.7, in particular at least 0.8, in particular at least 0.9. The upper limit for the ratio between the transition-cell length L3 and the free-cell length L2 (L3/L2) is preferably a value of at most 2.5, in particular at most 2.3, in particular at most 2.1, in particular at most 2, in particular at most 1.8, in particular at most 1.6, in particular at most 1.5.

For the ratio between the anchoring-cell length L1 and the connector spacing L4 (L1/L4), a value of at least 1, in particular at least 1.2, in particular at least 1.4, in particular at least 1.5 is preferably provided. Preferably, the ratio between anchoring-cell length L1 and connector spacing L4 (L1/L4) is at most 3, in particular at most 2.8, in particular at most 2.6, in particular at most 2.4.

The transition-cell length L3 can have a ratio to the connector spacing L4 (L3/L4) of at least 0.9, in particular at least 1, in particular at least 1.1, in particular at least 1.2. Preferably, the ratio between the transition-cell length L3 and the connector spacing L4 (L3/L4) is at most 2.5, in particular at most 2.4, in particular at most 2.3, in particular at most 2.2, in particular at most 2.1, in particular at most 2.0.

A lower limit for the ratio between the connector spacing L4 and the tip spacing L5 (L4/L5) is preferably at least 0.8, in particular at least 0.9, in particular at least 1, in particular at least 1.0. The maximum value for the ratio between the connector spacing L4 and the tip spacing L5 (L4/L5) is preferably at most 2.5, in particular at most 2.4, in particular at most 2.3, in particular at most 2.2, in particular at most 2.1, in particular at most 2.

The ratio between the start spacing L6 and the residual strut spacing L7 (L6/L7) is preferably at least 0.2, in particular at least 0.3, in particular at least 0.4, in particular at least 0.5. For the ratio between start spacing L6 and residual strut spacing L7 (L6/L7), a maximum value of at most 2, in particular at most 1.8, in particular at most 1.6, in particular at most 1.4, in particular at most 1.2, in particular at most 1, in particular at most 0.8, in particular at most 0.6 is preferably provided.

The ratio of the retaining element length L8 to the anchoring-cell length L1 (L8/L1) is preferably at least 0.2. The ratio between retaining element length L8 and anchoring-cell length L1 (L8/L1) is preferably at most 1.0, in particular at most 0.75, in particular at most 0.7, in particular at most 0.65, in particular at most 0.6. The aforementioned maximum values preferably also apply to the ratio between the opening length L9 and the anchoring-cell length L1. Specifically, the ratio between the opening length L9 and the anchoring-cell length L1 (L9/L1) is preferably at most 1.0, in particular at most 0.75, in particular at most 0.7, in particular at most 0.65, in particular at most 0.6. The lower limit for the ratio between the opening length L9 and the anchoring-cell length L1 (L9/L1) is preferably at least 0.2.

The ratio of the retaining element length L8 to the opening length L9 (L8/L9) is preferably at least 0.6, in particular at least 0.7, in particular at least 0.8, in particular at least 0.9, in particular at least 1. The upper limit for the ratio between retaining element length L8 and opening length L9 (L8/L9) is preferably at most 2.5, in particular at most 2.2, in particular at most 2.0, in particular at most 1.8, in particular at most 1.6, in particular at most 1.5.

The connector spacing L4, the tip spacing L5, the start spacing L6, the residual strut spacing L7, the retaining element length L8 and the opening length L9 can in each case relate to different configurations of the closed cells 13, in particular both to the anchoring cell 13a and also to the free cell 13b and also the transition cell 13c.

FIG. 13a shows the stent from FIG. 11 in the implanted state within a hollow organ 40 of the body. The stent or the mesh structure 12 is expanded in the hollow organ 40 of the body and bears on the vessel walls of the hollow organ 40. FIG. 13 also shows the tip of a delivery system 30, with which the stent has been introduced into the hollow organ 40 of the body. It can be clearly seen from FIG. 13a that all the retaining elements 14 of the mesh structure 12 and their tips 17 point in the distal direction, that is to say away from the delivery system 30. In this way, it is made possible to draw the stent or the mesh structure 12 back into the delivery system 30, particularly if the mesh structure 12 is only partially deployed from the delivery system 30. By means of the retaining elements 14 being oriented in the same direction, particularly in the distal direction, it is possible to prevent the retaining elements 14 from catching when drawn back into the delivery system 30.

The anchoring of the medical device or of the mesh structure 12 in a hollow organ 40 of the body, particularly in a blood vessel, takes place mainly through the geometric relationships in the cells 13 that have retaining elements 14. The improved anchoring in the blood vessel or generally in the body cavity 40 is achieved by suitable configuration of the first and second struts 11, 15, of the length of the cells 13, and of the wall thickness and strut widths. In particular, the degree of the radial deflection of the retaining elements 14 can be influenced by the geometrical configuration. The retaining elements 14 can be constructed in such a way that a deflection is effected only through a curving of the mesh structure 12 along a longitudinal axis.

Moreover, the fact that the retaining elements 14, in particular all the retaining elements 14, are oriented in the distal direction permits an improved anchoring of the mesh structure 12 or the implant in the body cavity 40. This advantage is of particular help when using the stent or the mesh structure 12 as a coil stent.

Coil stents are used to cover an aneurysm 50, as is shown in FIG. 13b. Generally, it has proven advantageous to guide a catheter through the cells 13 of the mesh structure 12 into the aneurysm 50, in order to introduce coils into the aneurysm 50. In doing this, there is the danger that the coil catheter becomes hooked in the mesh structure 12, and the mesh structure is displaced by an axial movement of the coil catheter in the hollow organ 40 of the body. However, the retaining elements 14 provide an additional anchoring of the mesh structure 12 in the vessel wall of the hollow organ 40, such that the risk of displacement or dislocation of the mesh structure 12 is reduced or minimized. The mesh structure 12 according to FIG. 11, which is shown in use as a coil catheter in FIG. 13b, is particularly suitable for covering aneurysms 50, since the retaining elements 14 are arranged in the area of the axial end portions of the mesh structure 12 that can be positioned outside the aneurysm 50, preferably in the healthy areas of the hollow organ 40.

Generally, the radial force of the mesh structure 12 has the effect that the struts 11 of the mesh structure 12 press at least partially into the vessel wall of the hollow organ 40. Through the radial force issuing from the mesh structure 12 and acting on the hollow organ 40, a kind of undercut is obtained, as is shown in FIG. 14a. In other words, the struts 11 of the mesh structure 12 are at least partially surrounded by the vessel wall of the hollow organ 40. In this way, an anchoring of the mesh structure 12 in the vessel wall of the hollow organ 40 is obtained. In particular, the aforementioned undercut works against an axial dislocation of the stent. This effect is intensified in closed cells 13 that have a retaining element 14, as can be seen in FIG. 14b. In addition to the first struts 11, the retaining elements 14 are also pressed at least partially into the vessel wall of the hollow organ 40 in the implanted state, thereby increasing the overall surface area of the mesh structure 12 pressing into the vessel wall. This leads to improved anchoring and greater resistance to axial dislocation.

The aforementioned effect is further intensified when the mesh structure 12 is arranged in curved vessel portions. FIG. 15a shows the anchoring of a closed cell 13 with a retaining element 14 in a vessel wall of a hollow organ 40. A depth of pressing-in E3 is obtained which reflects the amount by which the closed cell 13 is embedded or pressed into the vessel wall. When the closed cell 13 according to FIG. 15a is arranged in a curve of a vessel, as is shown in FIG. 15b, a depth of penetration E2 of the retaining element 14 is obtained which increases from the start point 26 as far as the tip 17 and is greater than the depth of pressing-in E1 of the first struts 11. Therefore, when arranged in a curve of a vessel, the retaining element 14 is deflected radially outward in relation to the first struts 11 or the closed cell 13. The anchoring of the mesh structure 12 in a curve of a vessel is thereby improved.

Preferably, the first struts 11 of the closed cell 13 and the second struts 15, 16 of the retaining element 14 have different strut widths. In particular, a ratio of the strut width S1 of the first struts 11 to the strut width S2 of the second struts 15, 16 (S1/S2) is preferably provided which is at least 0.5 and at most 2. Here, the strut width S1 of the first struts 11 and/or the strut width S2 of the second struts 15, 16 preferably covers a value of at least 0.010 mm, in particular at least 0.015 mm, in particular at least 0.020 mm, in particular at least 0.025 mm. The strut width S1 of the first struts 11 and/or the strut width S2 of the second struts 15, 16 can be at most 0.06 mm, in particular at most 0.08 mm, in particular at most 0.07 mm, in particular at most 0.06 mm.

The wall 10 of the implant preferably has a wall thickness W of at least 0.03 mm, in particular at least 0.04 mm, in particular at least 0.05 mm, and/or at most 0.09 mm, in particular at most 0.08 mm, in particular at most 0.07 mm, in particular at most 0.06 mm, in particular at most 0.055 mm. Advantageously, a ratio of the wall thickness W to the strut width S1 of the first struts 11 or to the strut width S2 of the second struts 15, 16 (W/S1 or W/S2) is provided which is at least 0.8, in particular at least 0.9, in particular at least 1, in particular at least 1.1, in particular at least 1.2, in particular at least 1.3, and/or at most 2.0, in particular at most 1.8, in particular at most 1.6, in particular at most 1.4.

In the expanded state or production state, the cross-sectional diameter of the implant or of the mesh structure 12 is preferably at least 1.5 mm, in particular at least 1.75 mm, in particular at least 2.0 mm, in particular at least 2.25 mm, in particular at least 2.5 mm. The upper limit provided for the expanded diameter D of the implant or of the mesh structure 12 is a value of at most 6.5 mm, in particular at most 5.5 mm, in particular at most 5.0 mm, in particular at most 4.5 mm, in particular at most 4.0 mm, in particular at most 3.5 mm, in particular at most 3.0 mm.

A preferred use diameter is also specified, which corresponds to the preferred diameter in the implanted state and is preferably at least 1.0 mm, in particular at least 1.5 mm, in particular at least 2.0 mm. The preferred use diameter can be at most 6.0 mm, in particular at most 5.5 mm, in particular at most 4.5 mm, in particular at most 4.0 mm, in particular at most 3.5 mm, in particular at most 2.5 mm. The stent length is preferably at least 10 mm, in particular at least 12 mm, in particular at least 14 mm, in particular at least 15 mm. A maximum length of the stent or a maximum stent length is preferably at most 150 mm, in particular at most 140 mm, in particular at most 130 mm, in particular at most 120 mm, in particular at most 100 mm, in particular at most 80 mm, in particular at most 60 mm, in particular at most 40 mm, in particular at most 20 mm.

### LIST OF REFERENCE SIGNS

10 wall
11 first struts
11a strut start portion
11b residual strut portion
12 mesh structure
13 closed cell
13a anchoring cell
13b free cell
13c transition cell
14 retaining element
15a, 16a strut continuation
15, 16 second struts
17 tip
17a further tip
18 further cell
19 connector
20, 21 edge areas
22 intermediate area
23 ring segments
24 axial end
25 X-ray marker
26 start point
30 delivery system
40 hollow organ of body
50 aneurysm
L′ longitudinal axis
L″ longitudinal axis
L1 anchoring-cell length
L2 free-cell length
L3 transition-cell length
L4 connector spacing
L5 tip spacing
L6 start spacing
L7 residual strut spacing
L8 retaining element length
L9 opening length

E1 depth of penetration of first struts 11
E2 depth of penetration of retaining element 14
E3 depth of pressing-in
S1 strut width of the first struts 11
S2 strut width of the second struts 15, 16
W wall thickness

1. A medical implant with a wall, which comprises a mesh structure that can be transferred from a compressed state to an expanded state and that is formed by first struts, wherein the mesh structure has closed cells, each with a retaining element adapted to anchor the expanded mesh structure in a vessel, characterized in that the retaining element has at least two second struts which, at one end, are connected to each other and form a tip, which projects into the cell, and, at the other end, are connected to the first struts of the cell.

2. The implant as claimed in claim 1, characterized in that the second struts of the retaining element form, with the first struts of a cell of the mesh structure, a further cell, which is arranged in the cell of the mesh structure.

3. The implant as claimed in claim 2, characterized in that the shape of the further cell corresponds to the shape of the cell of the mesh structure.

4. The implant as claimed in claim 1, characterized in that the first struts of the mesh structure are connected to the second struts of the retaining element in each case between two connectors axially delimiting the first struts of the mesh structure.

5. The implant as claimed in claim 1, characterized in that the second struts of the retaining element are connected in each case to an axial end of the first struts of the mesh structure, wherein the retaining element replaces a connectoraxially delimiting the first struts.

6. The implant as claimed in claim 1, characterized in that a longitudinal axis L′ of the further cell is flush with a longitudinal axis L″ of the associated cell of the mesh structure.

7. The implant as claimed in claim 1, characterized in that the tip of the retaining element is arranged at least at the height of lateral connectors, which connect cells of the mesh structure that are arranged next to one another in the circumferential direction.

8. The implant as claimed in claim 1, characterized in that the tip points in the distal direction of the implant.

9. The implant as claimed in claim 1, characterized in that, in the longitudinal direction of the wall, at least one edge are, in particular two edge areas, and at least one intermediate area of the mesh structure are arranged in the longitudinal direction of the wall, wherein the retaining elements are formed in the edge area.

10. The implant as claimed in claim 1, characterized in that the closed cells of the mesh structure form a plurality of ring segments arranged one after another in the axial direction of the wall, wherein the cells of a ring segment have at least one retaining element, in particular all the cells of the ring segment each have a retaining element.

11. The implant as claimed in claim 10, characterized in that the number of the distally arranged ring segments with retaining elements is greater than the number of the proximally arranged ring segments with retaining elements.

12. The implant as claimed in claim 1, characterized in that the cell of the mesh structure comprises a diamond-shaped cell.

13. The implant as claimed in claim 1, characterized in that the further cell, which is arranged in the cell of the mesh structure, comprises a diamond-shaped cell.

14. The implant as claimed in claim 1, characterized in that the retaining element is adapted in such a way that, when the mesh structure curves along a longitudinal axis of the mesh structure, the tip of the retaining element automatically orients itself radially outward.

15. The implant as claimed in claim 1, characterized in that a plurality of retaining elements are provided, which are distributed across the circumference of the mesh structure, and, when the mesh structure curves along a longitudinal axis of the mesh structure, the tips of the retaining elements automatically orient themselves radially outward, in particular only the tips of the retaining elements arranged on a side of the mesh structure directed away from the center of curvature.

16. The implant as claimed in claim 1, characterized in that a plurality of retaining elements are provided, wherein the tips of all the retaining elements point in the same direction, in particular in the distal direction relative to a delivery system.
